(19) United States
(12) Patent Application Publication
(10) Pub. No.: US 2021/0085256 A1
(43) Pub. Date: Mar. 25, 2021

(54) TRAINING DEVICE, TRAINING METHOD, IDENTIFICATION DEVICE, IDENTIFICATION METHOD, AND RECORDING MEDIUM

(71) Applicant: CASIO COMPUTER CO., LTD., Tokyo (JP)

(72) Inventors: Mitsuyasu NAKAJIMA, Tokyo (JP); Kouichi NAKAGOME, Tokorozawa-shi (JP); Takashi YAMAYA, Tokyo (JP); Yasushi MAENO, Tokyo (JP)

(73) Assignee: CASIO COMPUTER CO., LTD., Tokyo (JP)

(21) Appl. No.: 16/986,379

(22) Filed: Aug. 6, 2020

(30) Foreign Application Priority Data
Sep. 19, 2019 (JP) 2019-170826
May 29, 2020 (JP) 2020-094915

(51) Int. Cl. A61B 5/00; G06N 20/10

(52) U.S. Cl. CPC A61B 5/7267; G06N 20/10; A61B 5/4064; A61B 5/4812

(57) ABSTRACT

An aspect of the disclosure relates to a training device including a memory storing a program, and at least one processor configured to execute the program stored in the memory, in which the processor is configured to acquire pulse wave data to which biological reaction information is imparted, extract a local maximum point of a baseline or a local minimum point of a baseline derived from the pulse wave data as an identification reference point and set a correct answer label for the identification reference point based on the biological reaction information, set an analysis window for the extracted identification reference point and determine a feature vector of the identification reference point in the analysis window, and train a discriminator that identifies a cyclic alternating pattern (CAP) indicating a periodic brain wave activity by training data including the feature vector and the correct answer label.

### PRIORITY CLAIM BASED ON EARLIER APPLICATION

### [Application Number] Japanese Patent Application No. 2020-094915

### [Application Date] May 29, 2020

### BACKGROUND

### 1. Technical Field

The present disclosure relates to a biological reaction identification technology.

### 2. Related Art

As a sleep state monitoring system for determining a sleep state, determination of a sleep stage (light sleep, deep sleep, REM sleep, non-REM sleep and the like) based on a brain wave has been widely used (for example, JP 2011-83393 A). In addition, a sleep monitoring system using body movement or a pulse wave easier to acquire than the brain wave has been proposed (for example, JP 2018-161432 A).

Meanwhile, even though the sleep stage is used as one sleep index, the sleep stage may not have a high correlation with sleep subjectivity indicating a degree of satisfaction of a subject with respect to sleep. As a sleep index considered to have a high correlation with sleep subjectivity, a cyclic alternating pattern (CAP) (periodic brain wave activity) defined based on the brain wave has been known (for example, Terzano MG, Parrino L, Spaggiari MC, Palomba V, Rossi M, Smerieri A, et al. CAP variables and arousals as sleep electroencephalogram markers for primary insomnia. Clin Neurophysiol 2003 September; 114(9): 1715-23.). In general, sleep quality is poor during a period in which CAP frequency is high.

### SUMMARY

To solve the above-mentioned problem, an aspect of the disclosure relates to a training device including a memory storing a program, and at least one processor configured to execute the program stored in the memory, in which the processor is configured to acquire pulse wave data to which biological reaction information is imparted, extract a local maximum point of a baseline or a local minimum point of a baseline derived from the pulse wave data as an identification reference point and set a correct answer label for the identification reference point based on the biological reaction information, set an analysis window for the extracted identification reference point and determine a feature vector of the identification reference point in the analysis window, and train a discriminator that identifies a cyclic alternating pattern (CAP) indicating a periodic brain wave activity by training data including the feature vector and the correct answer label.

Another aspect of the disclosure relates to an identification device including a memory storing a program and at least one processor configured to execute the program stored in the memory, in which the processor is configured to acquire pulse wave data, extract a local maximum point of a baseline or a local minimum point of a baseline derived from the pulse wave data as an identification reference point, set an analysis window for the extracted identification reference point and determine a feature vector of the identification reference point in the analysis window, and input the feature vector to a trained discriminator that identifies a CAP indicating a periodic brain wave activity and acquire a CAP identification result.

### BRIEF DESCRIPTION OF THE DRAWINGS

FIG. 1 is a schematic diagram illustrating a biological reaction identification process according to an embodiment of the disclosure;

FIG. 2 is a block diagram illustrating a hardware configuration of a training device and an identification device according to the embodiment of the disclosure;

FIG. 3 is a block diagram illustrating a functional configuration of the training device according to the embodiment of the disclosure;

FIGS. 4A and 4B are schematic diagrams illustrating preprocessing of pulse wave data according to the embodiment of the disclosure;

FIG. 5 is a schematic diagram illustrating an identification reference point extraction process according to the embodiment of the disclosure;

FIGS. 6A, 6B, and 6C are diagrams illustrating CAP subclass waveforms according to the embodiment of the disclosure;

FIG. 7 is a flowchart illustrating a training process according to the embodiment of the disclosure;

FIG. 8 is a block diagram illustrating a functional configuration of the identification device according to the embodiment of the disclosure;

FIG. 9 is a flowchart illustrating an identification process according to the embodiment of the disclosure; and

FIG. 10 is an evaluation result showing an identification performance of the identification device according to the embodiment of the disclosure.

### DETAILED DESCRIPTION

The following embodiment discloses a training device of a discriminator that identifies a cyclic alternating pattern (CAP) (periodic brain wave activity) from pulse wave data of a subject, and an identification device that identifies the CAP from the pulse wave data using the trained discriminator.

### SUMMARY OF THE PRESENT DISCLOSURE

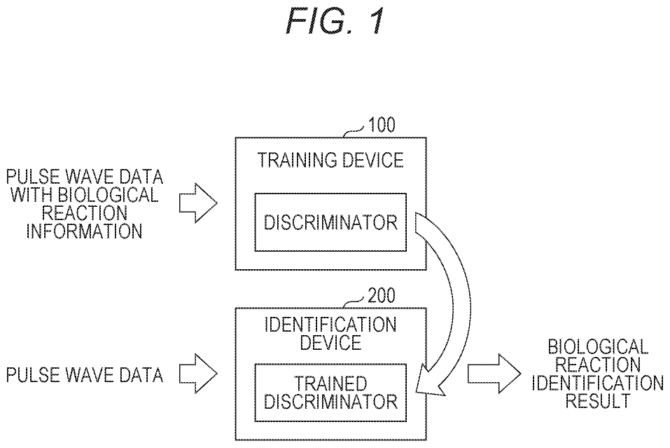

To outline the embodiment described below, as illustrated in FIG. 1, upon acquiring pulse wave data to which biological reaction information is imparted such as a CAP occurrence period, a training device 100 derives a baseline of the pulse wave data, and extracts a local maximum point of the derived baseline as an identification reference point. The training device 100 sets a correct answer label indicating the presence or absence or the type of a biological reaction based on biological reaction information for each identification reference point, sets an analysis window of different length for each identification reference point, and determines a feature vector including various statistical amounts in the set analysis window. The training device 100 uses training data including a pair of a feature vector and a correct answer label acquired for each identification reference point to train a discriminator that estimates a biological reaction identification result from the feature vector, and provides the trained discriminator to the identification device.

The identification device 200 inputs the input pulse wave data of the subject into the trained discriminator, and acquires an identification result of the biological reaction of the subject. In this way, instead of brain wave data which is relatively difficult to acquire, the identification device 200 may use a pulse wave sensor that is easy to wear to estimate a biological reaction such as a sleep state in consideration of sleep subjectivity of the subject based on pulse wave data collected in time series from the sleeping subject.

### [Hardware Configuration]

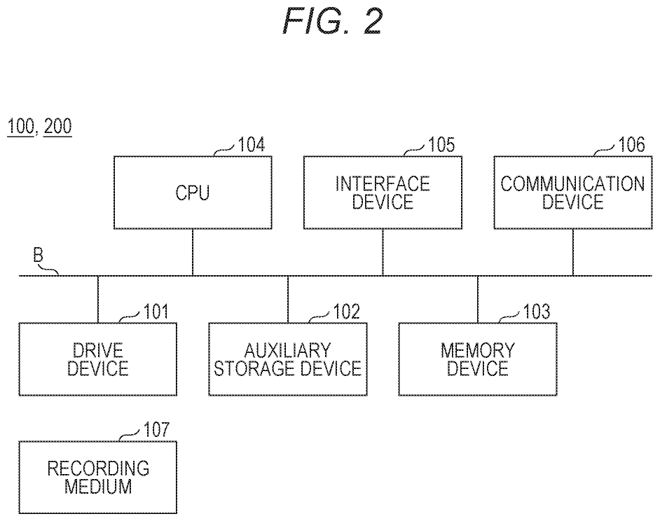

Here, the training device 100 and the identification device 200 may be, for example, a computing device such as a server, or may have a hardware configuration illustrated in FIG. 2. That is, each of the training device 100 and the identification device 200 includes a drive device 101, an auxiliary storage device 102, a memory device 103, a central processing unit (CPU) 104, an interface device 105, and a communication device 106 that are interconnected via a bus B.

Various computer programs including programs or instructions for realizing various functions and processes described below in the training device 100 and the identification device 200 may be provided by a recording medium 107 such as a compact disk-read only memory (CD-ROM). When the recording medium 107 storing a program is set in the drive device 101, the program is installed in the auxiliary storage device 102 from the recording medium 107 via the drive device 101. However, the program may not be installed by the recording medium 107, and may be downloaded from any external device via a network or the like. The auxiliary storage device 102 stores the installed program and stores necessary files, data and the like. The memory device 103 reads and stores a program or data from the auxiliary storage device 102 when a program activation instruction is given. The auxiliary storage device 102 and the memory device 103 are implemented as a non-transitory computer-readable storage medium that stores programs or instructions. The CPU 104, which functions as a processor, executes various functions and processes of the training device 100 and the identification device 200 according to various data such as programs stored in the memory device 103 or parameters necessary to execute the programs. The interface device 105 is used as a communication interface for connecting to a network or an external device. The communication device 106 executes various communication processes for communicating with an external device.

However, the training device 100 and the identification device 200 are not limited to the hardware configuration described above, and may be implemented by, for example, any other suitable hardware configuration such as one or more circuits that implement one or more of the functions and processes of the training device 100 and the identification device 200. For example, the training device 100 and the identification device 200 may be implemented as a wristwatch-type wearable type device worn on an arm of an examinee, an earphone-type hearable type device inserted into an ear of the examinee, or a smartphone.

### [Training Device]

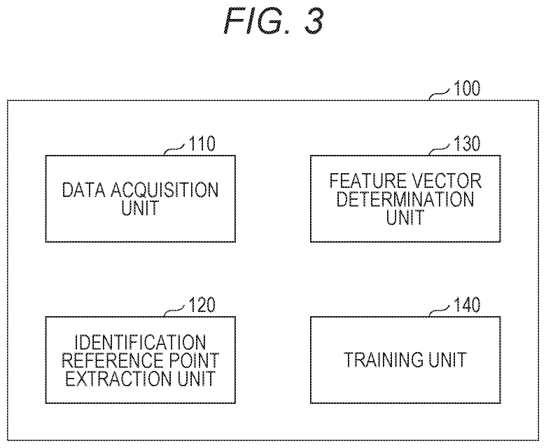

Next, a description will be given of the training device 100 according to the embodiment of the disclosure with reference to FIG. 3. FIG. 3 is a block diagram illustrating a functional configuration of the training device 100 according to the embodiment of the disclosure.

As illustrated in FIG. 3, the training device 100 includes a data acquisition unit 110, an identification reference point extraction unit 120, a feature vector determination unit 130, and a training unit 140.

The data acquisition unit 110 acquires pulse wave data to which biological reaction information is imparted. For example, a sleep polygraphy device and a pulse wave sensor are attached to the subject, and brain wave data and pulse wave data during sleep are acquired at the same time. An engineer or the like analyzes the acquired brain wave data, identifies a biological reaction such as a CAP, and imparts an occurrence period of the identified biological reaction to the pulse wave data. In this way, the pulse wave data to which the biological reaction information is imparted is generated.

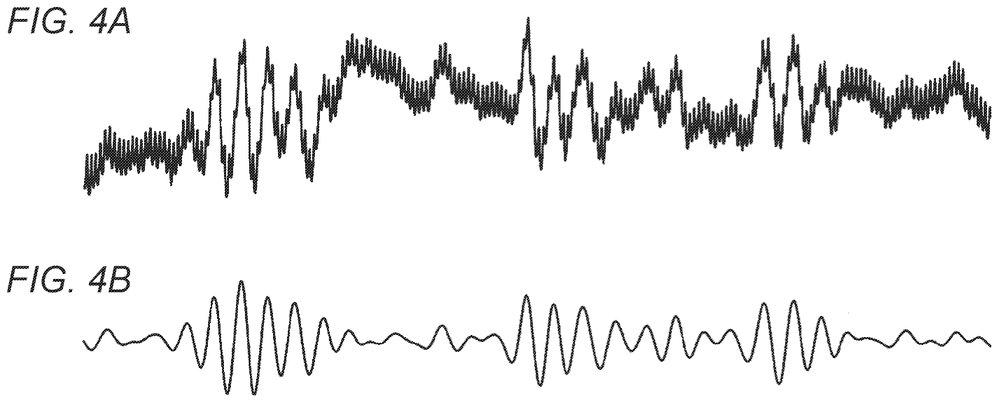

Here, the pulse wave data is collected from a sleeping subject by a pulse wave sensor or a wearable device equipped with the pulse wave sensor. For example, the pulse wave data may be pulse wave data illustrated in FIG. 4A. Here, a vertical axis represents output from the pulse wave sensor, and a horizontal axis represents time. The output of the pulse wave sensor repeatedly increases and decreases at a cycle of about 1 second, and corresponds to a pulse. For example, the pulse wave sensor may be realized by various sensors such as is a sensor that detects a volume pulse wave by a photoplethysmography (PPG) method, a Doppler blood flow meter that detects a blood flow pulse wave, and a piezoelectric sensor that detects a pressure pulse wave.

In one embodiment, the data acquisition unit 110 may acquire pulse wave data to which a CAP subclass occurrence period is imparted. The CAP can be subdivided into A1, A2, and A3 as subclasses due to differences in brain wave component. A change is composition ratio of the CAP subclass is considered to have a correlation with a sleep disorder. For example, a ratio of A3 increases in sleep apnea, and a ratio of A1 increases in sleepwalking and misidentification of sleep state. That is, when the CAP occurrence frequency and the CAP subclass composition ratio can be detected, it is possible to estimate a sleep state of the subject in more detail.

The data acquisition unit 110 provides the acquired pulse wave data to which biological reaction information is imparted to the identification reference point extraction unit 120.

The identification reference point extraction unit 120 extracts the local maximum point of the baseline derived from the pulse wave data as an identification reference point, and sets the correct answer label on the identification reference point based on the biological reaction information. Specifically, the identification reference point extraction unit 120 first derives the baseline of the pulse wave data. For example, upon acquiring the pulse wave data illustrated in FIG. 4A from the data acquisition unit 110, the identification reference point extraction unit 120 performs bandpass filter processing on the acquired pulse wave data, and derives a baseline of the pulse wave data illustrated in FIG. 4B. For example, the bandpass filter processing may be processing for extracting, for example, a range of 0.08 Hz to 0.2 Hz for the pulse wave data acquired from the data acquisition unit 110.

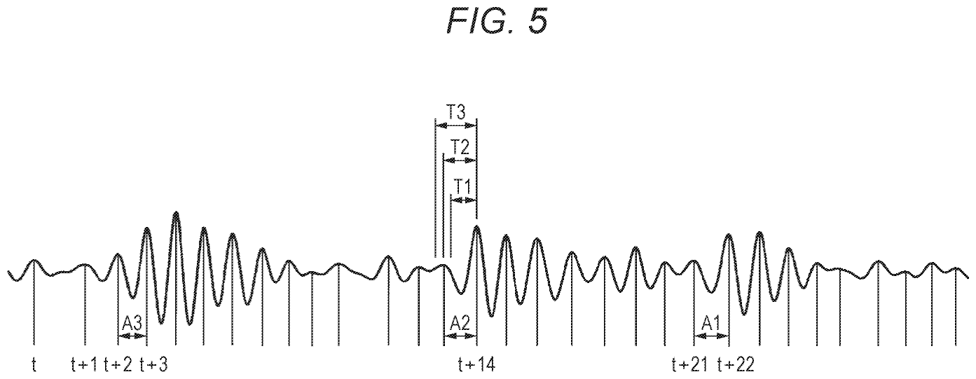

When the baseline is derived in this manner, the identification reference point extraction unit 120 further extracts a local maximum point of the derived baseline. For example, with respect to the baseline illustrated in FIG. 4B, the identification reference point extraction unit 120 extracts local maximum points t, t+1, t+2, . . . illustrated in FIG. 5, and holds the extracted local maximum point as identification reference points for setting the correct answer label indicating the biological reaction. CAP occurrence periods A3, A2, and A1 as the biological reaction information acquired by the data acquisition unit 110 are superimposed on the baseline illustrated in FIG. 5. Here, A3, A2, and A1 indicate CAP subclasses A3, A2, and A1, respectively. In general, the CAP includes Phase A and Phase B following Phase A, and it is known that a period of Phase A appears as a major change in the brain wave. In the pulse wave, a change in the pulse wave occurs near the period of Phase A. Therefore, the CAP occurrence period imparted to the pulse wave data can be regarded as the period of Phase A.

A pulse wave data acquisition system in the present embodiment is configured as a system in which a signal level rises when vasoconstriction occurs, and the above-mentioned identification reference point is the local maximum point. However, when the pulse wave data acquisition system is a system in which the signal level decreases when vasoconstriction occurs, a local minimum point becomes the identification reference point.

The identification reference point extraction unit 120 may determine the correct answer label set for the identification reference point based on an inclusion relationship between each identification reference point and the biological reaction occurrence period. For example, in a specific example illustrated in FIG. 5, the identification reference point extraction unit 120 may set a correct answer label of “without CAP” for the identification reference points t and t+1 not included in the CAP occurrence period, and set a correct answer label of “A3” for the identification reference points t+2 and t+3 included in the occurrence period of the CAP subclass A3. Similarly, the identification reference point extraction unit 120 may set the correct answer label of “A2” for the identification reference points t+13 and t+14 included in the occurrence period of the CAP subclass A2, and set a correct answer label of “without CAP” for other identification reference points.

However, setting of the correct answer label according to the disclosure is not limited thereto. Even when the identification reference point is not included in the CAP period, a correct answer label of “with CAP” may be set for the identification reference point adjacent to the CAP period. For example, in the specific example illustrated in FIG. 5, even though the CAP subclass A1 does not include any of the identification reference points, the identification reference point extraction unit 120 may set the correct answer label of “A1” for one or both of the identification reference points t+21 and t+22 adjacent to the CAP subclass A1.

Further, in the above-mentioned embodiment, the four correct answer labels of “without CAP”, “A1”, “A2”, and “A3” are provided. However, the correct answer labels according to the disclosure are not limited thereto, and it is possible to use an appropriate number of correct answer labels depending on the biological reaction of a detection target.

The identification reference point extraction unit 120 provides the identification reference points to which the correct answer labels are imparted to the feature vector determination unit 130 and the training unit 140.

The feature vector determination unit 130 sets an analysis window for the extracted identification reference point and determines the feature vector of the identification reference point in the analysis window. Here, each component of the feature vector may be various statistical amounts of pulse wave data calculated in a period defined by one or more analysis windows set for each identification reference point. For example, the feature vector may be [W1_m, W2_sd, W2_max, W3_max, W4_mm, W5_mm]. Here, W1_m is an average of a pulse wave baseline in an analysis window W1, W2_sd and W2_max are a standard deviation and a maximum value of a pulse wave baseline in an analysis window W2, respectively, W3_max is a maximum value of a pulse wave baseline in an analysis window W3, and W4_mm and W5_mm are maximum/minimum differences between pulse wave baselines in the analysis windows W4 and W5, respectively. In this way, the analysis window is variably set according to the waveform of the pulse wave data by utilizing the local maximum point of the baseline, instead of a fixed time interval. Further, a plurality of analysis windows having different lengths may be set for each identification reference point.

In one embodiment, the feature vector determination unit 130 may set the analysis window so that two identification reference points become a start point and an end point. For example, for an identification reference point t+1, the feature vector determination unit 130 may set a period from an identification reference point t+1 to an identification reference point t+2 as an analysis window. Alternatively, the feature vector determination unit 130 may set a period from the identification reference point t+1 to an identification reference point t+3 as an analysis window for the identification reference point t+1. Alternatively, the feature vector determination unit 130 may set a period from an identification reference point t to the identification reference point t+1 as an analysis window for the identification reference point t+1. Alternatively, the feature vector determination unit 130 may set a period from the identification reference point t+2 to the identification reference point t+3 as an analysis window for the identification reference point t+1.

In addition, in one embodiment, the feature vector determination unit 130 may set the analysis window so that one identification reference point becomes a start point and a local minimum point between identification reference points adjacent to the start point becomes an end point. For example, for the identification reference point t+1, the feature vector determination unit 130 may identify a local minimum point in a section from the identification reference point t+2 adjacent to the identification reference point t+1 to the identification reference point t+3, and set a period from the identification reference point t+1 to the local minimum point as an analysis window. Alternatively, for the identification reference point t+1, the feature vector determination unit 130 may set an analysis window so that the identification reference point t adjacent to the identification reference point t+1 is used as a start point, and the local minimum point in the section from the identification reference point t+2 adjacent to the identification reference point t+1 to the identification reference point t+3 is used as an end point. Alternatively, for the identification reference point t+1, the feature vector determination unit 130 may set an analysis window so that the identification reference point t adjacent to the identification reference point t+1 is used as a start point, and a local minimum point in a section from the identification reference point t+1 to the adjacent identification reference point t+2 is used as an end point.

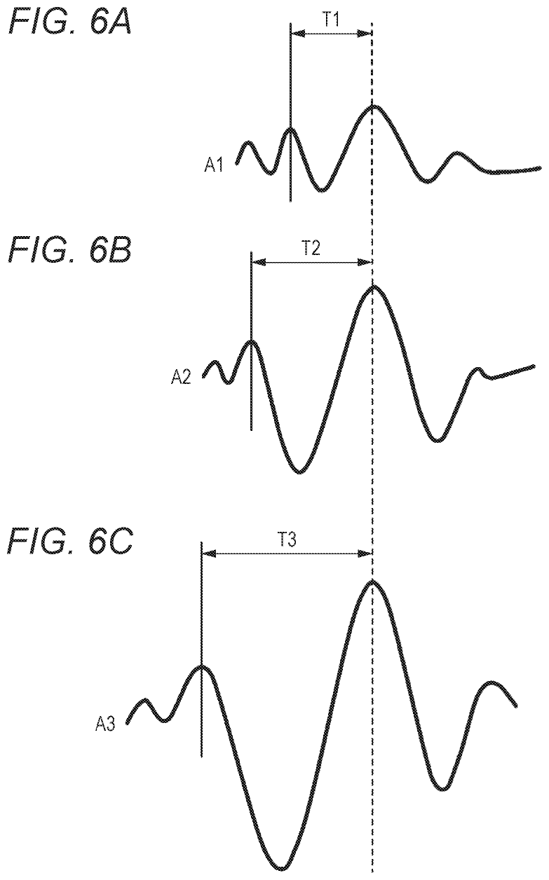

Further, in one embodiment, the feature vector determination unit 130 may set the analysis window based on a waveform of the biological reaction. For example, the CAP subclasses A1, A2, and A3 have waveforms illustrated in FIGS. 6A to 6C, respectively. Analysis windows having lengths T1, T2, and T3 may be set according to these waveforms. For example, from the pulse wave data to which the occurrence period of the CAP subclass acquired by the data acquisition unit 110 is imparted, as the average value of Phase A of each CAP subclass, it is presumed that A1 is 6.5 seconds, A2 is 7.5 seconds, and A3 is 11 seconds. In this case, T1=6.5 seconds, T2=7.5 seconds, and T3=11 seconds are set. Here, the analysis windows having different lengths may be set in any of front and back directions with respect to a time axis. For example, for the identification reference point t+14 illustrated in FIG. 5, the feature vector determination unit 130 may set the analysis windows of the periods of T1, T2, and T3 in a direction before t+14 with respect to the time axis, or set the analysis windows of the periods of T1, T2, and T3 in a direction after t+14 with respect to the time axis.

For example, for the feature vector [W1_m, W2_sd, W2_max, W3_max, W4_mm, W5_mm] described above, W1 may be an analysis window set so that the above-mentioned two identification reference points become the start point and the end point, W2 may be an analysis window set so that the above-mentioned one identification reference point becomes the start point, and the local minimum point between the identification reference points adjacent to the start point becomes the end point, and W3, W4, and W5 may be analysis windows of the periods of T1, T2, and T3 corresponding to the above-mentioned CAP subclasses A1, A2, and A3, respectively. Note that the components of the feature vector are not limited to the above or other statistical amounts, and may be any other appropriate feature amounts expressing the pulse waves in the analysis window.

Using the analysis window set in this way, the feature vector determination unit 130 calculates each component of the feature vector of each identification reference point and provides the calculated feature vector to the training unit 140.

The training unit 140 trains the discriminator by training data including a feature vector and a correct answer label. The discriminator is any suitable machine learning model that estimates an identification result of a biological reaction such as CAP subclass generation from the feature vector, and can be realized by, for example, a support vector machine, a logistic regression model, a neural network, a decision tree and the like. The training unit 140 uses the feature vector and correct answer label acquired for each identification reference point as training data to train the discriminator by any appropriate algorithm according to the type of the discriminator. For example, in a case where the discriminator is implemented by a support vector machine, the training unit 140 learns a feature vector group of training data in a feature space on the basis of obtaining a maximized hyperplane that maximizes a distance from the training data when determining a separating hyperplane by a correct answer label. Since training process of the support vector machine is well known, details thereof are omitted.

When a predetermined termination condition is satisfied, for example, when a training process is completed for all the training data provided from the identification reference point extraction unit 120 and the feature vector determination unit 130, the training unit 140 provides a finally acquired discriminator to the identification device 200 as a trained discriminator.

### [Training Process]

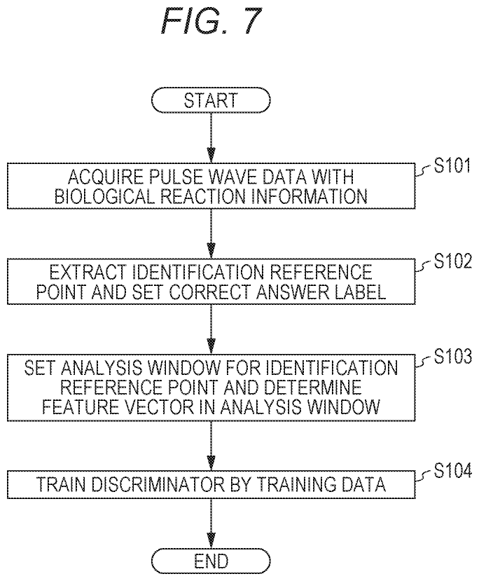

Next, a description will be given of a training process according to the embodiment of the disclosure with reference to FIG. 7. The training process is executed by the above-described training device 100, and may be realized by, for example, the processor of the training device 100 executing a program or an instruction. FIG. 7 is a flowchart illustrating the training process according to the embodiment of the disclosure.

As illustrated in FIG. 7, in step S101, the training device 100 acquires pulse wave data with biological reaction information. Specifically, the training device 100 acquires pulse wave data with biological reaction information created by analysis of brain wave data by an engineer or the like based on the brain wave data and the pulse wave data simultaneously collected from the subject. For example, the biological reaction is not limited, and may be related to a sleep state of the subject such as the CAP or brain wave awakening.

In step S102, the training device 100 extracts the local maximum point of the baseline derived from the pulse wave data as an identification reference point, and sets a correct answer label indicating a biological reaction to be identified at each identification reference point. Specifically, the training device 100 derives a baseline by performing bandpass filter processing on the pulse wave data, and extracts a local maximum point of the derived baseline as an identification reference point. When identification reference points for identifying the biological reaction are extracted in this way, the training device 100 imparts a correct answer label indicating an identification result of the biological reaction to each identification reference point. For example, the training device 100 may impart the correct answer label based on an inclusion relation between the identification reference point and the biological reaction occurrence period. Specifically, when the identification reference point is within the biological reaction occurrence period, the training device 100 may impart a correct answer label indicating occurrence of the corresponding biological reaction to the identification reference point. On the other hand, when the identification reference point is outside the biological reaction occurrence period, the training device 100 may impart a correct answer label indicating that the corresponding biological reaction has not occurred to the identification reference point. However, imparting of the correct answer label by the disclosure is not limited thereto, and any other appropriate imparting method may be applied.

In step S103, the training device 100 sets an analysis window for each identification reference point and determines a feature vector in the analysis window. For example, the training device 100 may set the analysis window so that two identification reference points become a start point and an end point. Alternatively, the training device 100 may set the analysis window so that one identification reference point becomes a start point and a local minimum point between identification reference points adjacent to the start point becomes an end point. Alternatively, the training device 100 may set the analysis window based on the waveform of the biological reaction. To determine each component of the feature vector, the training device 100 uses the analysis window set for the component to calculate the statistical amount of the pulse wave data in the analysis window.

In step S104, the training device 100 uses a pair of the feature vector and the correct answer label determined for each identification reference point as training data to train the discriminator that estimates the biological reaction from the feature vector. For example, the discriminator can be realized as a support vector machine, a logistic regression model, a neural network, a decision tree or the like. The training device 100 may train the discriminator according to any known algorithm depending on the realization mode of the discriminator. The training device 100 provides the finally acquired discriminator to the identification device 200.

### [Identification Device]

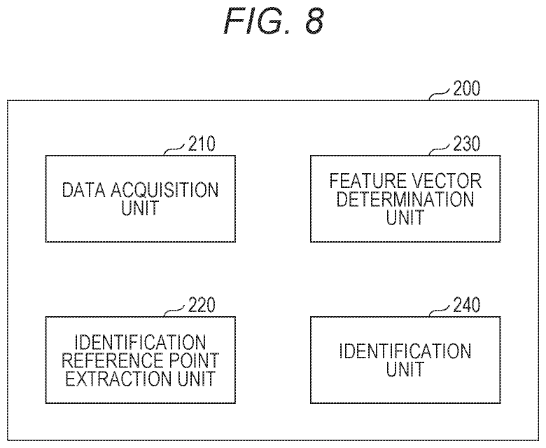

Next, a description will be given of the identification device 200 according to the embodiment of the disclosure with reference to FIG. 8. FIG. 8 is a block diagram illustrating a functional configuration of the identification device 200 according to the embodiment of the disclosure.

As illustrated in FIG. 8, the identification device 200 includes a data acquisition unit 210, an identification reference point extraction unit 220, a feature vector determination unit 230, and an identification unit 240.

The data acquisition unit 210 acquires pulse wave data. Specifically, the pulse wave data is collected from a sleeping subject by a pulse wave sensor or a wearable device equipped with the pulse wave sensor. For example, the pulse wave sensor may be realized by various sensors such as is a sensor that detects a volume pulse wave by a photoplethysmography (PPG) method, a Doppler blood flow meter that detects a blood flow pulse wave, and a piezoelectric sensor that detects a pressure pulse wave. Preferably, the pulse wave data may be collected by a pulse wave sensor of the same type as that of the pulse wave sensor used in the training process. Alternatively, when a different type of pulse wave sensor is used, any appropriate correction may be performed on the collected pulse wave data.

The identification reference point extraction unit 220 extracts a local maximum point of a baseline derived from the pulse wave data as an identification reference point. Specifically, the identification reference point extraction unit 220 first executes bandpass filter processing on the pulse wave data to derive the baseline of the pulse wave data. Then, the identification reference point extraction unit 220 further extracts a local maximum point of the derived baseline as an identification reference point, and provides the extracted local maximum point to the feature vector determination unit 230.

The feature vector determination unit 230 sets an analysis window for the extracted identification reference point and determines a feature vector of the identification reference point in the analysis window. Where the feature vector is data for input to the trained discriminator, the statistical amount and the analysis window of each component of the feature vector are defined in advance for the trained discriminator, and the feature vector determination unit 230 calculates the statistical amount of each component in the analysis window defined in advance.

For example, when the feature vector input to the trained discriminator is defined as [W1_m, W2_sd, W2_max, W3_max, W4_mm, W5_mm], the feature vector determination unit 230 determines each analysis window of W1 to W5 with respect to each identification reference point, and calculates the statistical amount of pulse wave data in each determined analysis window. That is, the feature vector determination unit 230 determines the analysis window W1 in which two identification reference points become a start point and an end point, and calculates an average W1_m of the pulse wave baselines in the analysis window W1. In addition, the feature vector determination unit 230 determines an analysis window W2 in which one identification reference point becomes the start point and a local minimum point between the start point and an adjacent identification reference point becomes the end point, and calculates a standard deviation W2_sd and a maximum value W2_max of the pulse wave baseline in the analysis window W2. Further, the feature vector determination unit 230 determines analysis windows of the length T1, T2, and T3 set based on the waveforms of the biological reaction, and calculates a maximum value W3_max of the pulse wave baseline in the analysis window W3, a maximum/minimum difference W4_mm of the pulse wave baseline in the analysis window W4, and a maximum/minimum difference W5_mm of the pulse wave baseline in the analysis window W5. The feature vector determination unit 230 provides the calculated feature vector to the identification unit 240.

The identification unit 240 inputs the feature vector to the trained discriminator and acquires an identification result. Specifically, the identification unit 240 identifies a biological reaction from a feature vector using a trained discriminator that identifies a biological response such as occurrence of a CAP subclass from the feature vector. The discriminator is any suitable machine learning model that estimates an identification result of a biological reaction such as CAP subclass generation from the feature vector, and can be realized by, for example, a support vector machine, a logistic regression model, a neural network, a decision tree and the like.

### [Identification Process]

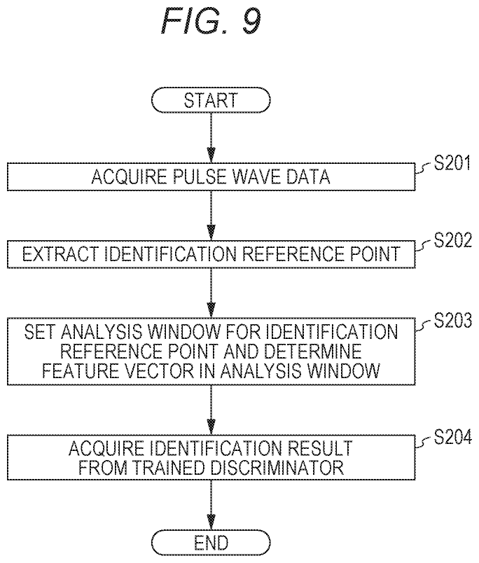

Next, a description will be given of an identification process according to the embodiment of the disclosure with reference to FIG. 9. The identification process is executed by the above-described identification device 200, and may be realized by, for example, the processor of the identification device 200 executing a program or an instruction. FIG. 9 is a flowchart illustrating the identification process according to the embodiment of the disclosure.

As illustrated in FIG. 9, in step S201, the identification device 200 acquires pulse wave data. Specifically, the pulse wave data is collected from a sleeping subject by a pulse wave sensor or a wearable device equipped with the pulse wave sensor. For example, the pulse wave data may be collected by a pulse wave sensor of the same type as that of the pulse wave sensor used in the training process. Alternatively, when a different type of pulse wave sensor is used, any appropriate correction may be performed on the collected pulse wave data.

In step S202, the identification device 200 extracts a local maximum point of a baseline derived from the pulse wave data as an identification reference point. For example, the identification device 200 may perform bandpass filter processing on the pulse wave data to derive the baseline.

In step S203, the identification device 200 sets an analysis window for each identification reference point and determines a feature vector in the analysis window. The statistical amount and the analysis window of each component of the feature vector are defined in advance for the trained discriminator, and the identification device 200 determines a predefined analysis window, and calculates a statistical amount of pulse wave data in the analysis window determined for each component.

In step S204, the identification device 200 inputs the feature vector to the trained discriminator and acquires an identification result. For example, when the trained discriminator is a model for identifying occurrence of a CAP subclass at the identification reference point from the feature vector at the identification reference point of the pulse wave data, the trained discriminator outputs an identification result of “without CAP”, “A1”, “A2” or “A3” from the input feature vector.

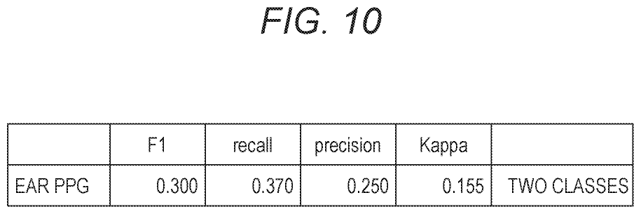

A table of FIG. 10 is an evaluation result showing an identification performance of a result of performing training using pulse wave data acquired from ears of a plurality of examinees (eight people in this evaluation) and identification of two classes with and without CAP using the identification device having the discriminator trained by the training device that trains the discriminator for identifying CAP (periodic brain wave activity) according to the invention. Note that Leave-one-out cross-validation (LOOCV) is used to evaluate the identification performance.

In this table, recall (sensitivity) is a numerical value indicating a proportion of identifiable ones to correct answers, and precision (precision rate) is a numerical value indicating a proportion of correct answers to identified ones. Recall and precision are contradictory to each other depending on the parameter. For example, an attempt to improve recall decreases precision. When the numerical value of recall is improved to identify the correct answers as many as possible, incorrect answers are included in the identified ones, that is, precision decreases. Depending on the use, recall is intentionally set high to prevent overlooking.

On the other hand, F1 is a numerical value for observing the overall identification performance, and is calculated by a harmonic mean of recall (sensitivity) and precision (precision rate).

In addition, similarly to F1, kappa is commonly used as a numerical value indicating the overall identification performance. The numerical value of kappa indicates that identification is not an accidental result (generally, when the numerical value is 0 or more, it may not be coincidence).

As can be seen from this table, by using the identification device having the discriminator trained by the training device of the invention, the CAP identification becomes possible within a practical range.

In addition, with regard to the identification performance, this evaluation is the result of only eight people (seven people in actual learning due to LOOCV), and it is considered that the identification performance is further improved when the number of trainees increases.

Note that even though the local maximum point of the baseline of the pulse wave data is extracted as the identification reference point in the above-described embodiment, the identification reference point according to the disclosure is not limited thereto and may be a local minimum point. For example, in the case of detecting a biological reaction due to blood vessel expansion, a local minimum point can be effectively used as an identification reference point.

In addition, when brain wave awakening is analyzed as a biological reaction, the disclosure may be applied to a two-class classification problem of the presence or absence of the brain wave awakening. In this instance, T1, T2, and T3 described above may be set based on a brain wave awakening period. For example, when a distribution of the brain wave awakening period is uniformly distributed over 3 to 15 seconds, a plurality of Ti's may be set every one second in 2 to 15 seconds. Alternatively, when the distribution of the brain wave awakening period is concentrated around about 5 seconds, only a specified value of 5 seconds may be used.

The invention of the present application is not limited to the above-described embodiments, and can be variously modified at the stage of implementation without departing from the spirit of the invention. In addition, the respective embodiments may be combined as appropriate as much as possible and implemented, and in this case, combined effects can be obtained. Further, the embodiments include inventions at various stages, and various inventions can be extracted by appropriately combining a plurality of disclosed constituent elements. For example, even when some constituent elements are deleted from all the constituent elements shown in the embodiment, the problems described in the section of the problem to be solved by the invention can be solved. When the effects described in the section of the effects of the invention can be obtained, a configuration in which the constituent elements are deleted can be extracted as the invention.

What is claimed is:

1. A training device comprising:
a memory storing a program; and
at least one processor configured to execute the program stored in the memory,
wherein the processor is configured to:
acquire pulse wave data to which biological reaction information is imparted;
extract a local maximum point of a baseline or a local minimum point of a baseline derived from the pulse wave data as an identification reference point and set a correct answer label for the identification reference point based on the biological reaction information;
set an analysis window for the extracted identification reference point and determine a feature vector of the identification reference point in the analysis window; and
train a discriminator that identifies a cyclic alternating pattern (CAP) indicating a periodic brain wave activity by training data including the feature vector and the correct answer label.

2. The training device according to claim 1, wherein the processor is configured to set the analysis window so that two identification reference points become a start point and an end point.

3. The training device according to claim 1, wherein the processor is configured to set the analysis window so that one identification reference point becomes a start point, and a local minimum point between identification reference points adjacent to the start point becomes an end point when a local maximum point of the baseline is extracted as the identification reference point, and set the analysis window so that one identification reference point becomes a start point, and a local maximum point between identification reference points adjacent to the start point becomes an end point when a local minimum point of the baseline is extracted as the identification reference point.

4. The training device according to claim 1, wherein the processor is configured to set the analysis window based on a waveform of a biological reaction.

5. The training device according to claim 1, wherein each component of the feature vector indicates a statistical amount calculated from an analysis window set for the component.

6. The training device according to claim 1,
wherein the processor is configured to acquire pulse wave data to which an occurrence period of a CAP subclass is imparted, and
the discriminator identifies occurrence of the CAP subclass.

7. The training device according to claim 1, wherein the processor is configured to determine a correct answer label set for the identification reference point based on an inclusion relationship between the identification reference point and an occurrence period of a biological reaction.

8. The training device according to claim 1, wherein the discriminator is realized by a support vector machine.

9. A training method performed by at least one processor, comprising the steps of:
acquiring pulse wave data to which biological reaction information is imparted;
extracting a local maximum point of a baseline or a local minimum point of a baseline derived from the pulse wave data as an identification reference point and setting a correct answer label for the identification reference point based on the biological reaction information;
setting an analysis window for the extracted identification reference point and determining a feature vector of the identification reference point in the analysis window; and
training a discriminator that identifies a CAP indicating a periodic brain wave activity by training data including the feature vector and the correct answer label.

10. A non-transitory computer-readable recording medium comprising a program stored thereon, which, when executed on at least one processor in a computer of a training device, causes the computer to:
acquire pulse wave data to which biological reaction information is imparted;
extract a local maximum point of a baseline or a local minimum point of a baseline derived from the pulse wave data as an identification reference point and set a correct answer label for the identification reference point based on the biological reaction information;
set an analysis window for the extracted identification reference point and determine a feature vector of the identification reference point in the analysis window; and
train a discriminator that identifies a CAP indicating a periodic brain wave activity by training data including the feature vector and the correct answer label.

11. An identification device comprising:
a memory storing a program; and
at least one processor configured to execute the program stored in the memory, wherein
the processor is configured to:
acquire pulse wave data,
extract a local maximum point of a baseline or a local minimum point of a baseline derived from the pulse wave data as an identification reference point,
set an analysis window for the extracted identification reference point and determine a feature vector of the identification reference point in the analysis window, and
input the feature vector to a trained discriminator that identifies a CAP indicating a periodic brain wave activity and acquire a CAP identification result.

12. The identification device according to claim 11, wherein the processor is configured to set the analysis window so that two identification reference points become a start point and an end point.

13. The identification device according to claim 11, wherein the processor is configured to set the analysis window so that one identification reference point becomes a start point, and a local minimum point between identification reference points adjacent to the start point becomes an end point when a local maximum point of the baseline is extracted as the identification reference point, and set the analysis window so that one identification reference point becomes a start point, and a local maximum point between identification reference points adjacent to the start point becomes an end point when a local minimum point of the baseline is extracted as the identification reference point.

14. The identification device according to claim 11, wherein the processor is configured to set the analysis window based on a waveform of a biological reaction.

15. The identification device according to claim 11, wherein each component of the feature vector indicates a statistical amount calculated from an analysis window set for the component.

16. The identification device according to claim 11, wherein the discriminator identifies occurrence of a CAP subclass.

17. The identification device according to claim 11, wherein the discriminator is realized by a support vector machine.

18. An identification method performed by at least one processor, comprising the steps of:
acquiring pulse wave data;
extracting a local maximum point of a baseline or a local minimum point of a baseline derived from the pulse wave data as an identification reference point;
setting an analysis window for the extracted identification reference point and determining a feature vector of the identification reference point in the analysis window; and
inputting the feature vector to a trained discriminator that identifies a CAP indicating a periodic brain wave activity and acquiring a CAP identification result.

19. A non-transitory computer-readable recording medium comprising a program stored thereon, when executed on at least one processor in a computer of an identification device, causes the computer to:
acquire pulse wave data;
extract a local maximum point of a baseline or a local minimum point of a baseline derived from the pulse wave data as an identification reference point;
set an analysis window for the extracted identification reference point and determine a feature vector of the identification reference point in the analysis window; and
input the feature vector to a trained discriminator that identifies a CAP indicating a periodic brain wave activity and acquire a CAP identification result.
